(19) United States
(12) Patent Application Publication
(10) Pub. No.: US 2023/0385731 A1
(43) Pub. Date: Nov. 30, 2023

(54) TASK MANAGEMENT TOOL

(71) Applicant: Citrix Systems, Inc., Ft. Lauderdale, FL (US)

(72) Inventors: Zongpeng Qiao, Nanjing (CN); Ze Chen, Nanjing (CN); Wenshuang Zhang, Nanjing (CN); Xuan Liu, Nanjing (CN)

(73) Assignee: Citrix Systems, Inc., Ft. Lauderdale, FL (US)

(21) Appl. No.: 17/847,678

(22) Filed: Jun. 23, 2022

Related U.S. Application Data
(63) Continuation of application No. PCT/CN2022/095815, filed on May 28, 2022.

(51) Int. Cl. G06Q 10/06; G16H 20/70; A61B 5/16; A61B 5/00

(52) U.S. Cl. CPC G06Q 10/06316; G06Q 10/06398; G06Q 10/063114; G16H 20/70; A61B 5/165; A61B 5/4815

(57) ABSTRACT

A task management process includes receiving, by a processor and from a task management service, one or more tasks to be performed by a user; computing, by the processor, a task score for each of the one or more tasks to be performed by the user; determining, by the processor, a mood status associated with the user; comparing, by the processor, the mood status to the task score for each of the one or more tasks to be performed by the user; determining, by the processor and based on the comparison, a recommended task from among each of the one or more tasks to be performed by the user; and sending, by the processor, the recommended task to the task management service for display to the user.

### BACKGROUND

Project management tools facilitate the planning, organization, and management of tasks within a given set of constraints. For example, project management software, such as Wrike by Wrike, Inc. of San Jose, California, can be used to optimize the allocation of resources—including human labor—to achieve project deliverables within predefined scope, time, and budget constraints. Typically, such tools focus on objective measures of achieving certain goals within the given constraints, such as the cost and availability of the resources needed to complete each step of the project under ideal or otherwise anticipated conditions. When contingencies arise due to deviations from the expected conditions, the tools can help recalibrate the tasks needed to minimize negative impacts to the project. Existing tools often rely on mileposts, or checkpoints, to help individuals and team members continuously or periodically assess progress and schedule additional tasks for achieving subsequent mileposts. Ultimately, the success of a project depends on the ability of the project members to complete all of the planned tasks within the given constraints.

### SUMMARY

One example provides a task management method including receiving, by a processor and from a task management service, one or more tasks to be performed by a user; computing, by the processor, a task score for each of the one or more tasks to be performed by the user; determining, by the processor, a mood status associated with the user; comparing, by the processor, the mood status to the task score for each of the one or more tasks to be performed by the user; determining, by the processor and based on the comparison, a recommended task from among each of the one or more tasks to be performed by the user; and sending, by the processor, the recommended task to the task management service for display to the user. In some examples, computing the task score includes computing a task familiarity score for one or more tasks completed by the user and computing a task difficulty score for the one or more tasks completed by the user. In some examples, the task familiarity score is based at least in part on a term frequency-inverse document frequency matrix representing words in a given task description. In some examples, the task difficulty score is based at least in part on a difference between an estimated effort to complete the one or more tasks completed by the user and an actual effort to complete the one or more tasks completed by the user. In some examples, determining the mood status includes receiving, from the task management service, a mood status manually selected by the user via a graphical user interface of the task management service. In some examples, determining the mood status includes receiving, from a smart device, health data representing a physiological indication of the user and/or a physical activity of the user. In some examples, the health data includes a sleep duration of the user.

Another example provides a computer program product including one or more non-transitory machine-readable mediums having instructions encoded thereon that when executed by at least one processor cause a process to be carried out. The process includes receiving, from a task management service, one or more tasks to be performed by a user; computing a task score for each of the one or more tasks to be performed by the user; determining a mood status associated with the user; comparing the mood status to the task score for each of the one or more tasks to be performed by the user; determining, based on the comparison, a recommended task from among each of the one or more tasks to be performed by the user; and sending the recommended task to the task management service for display to the user. In some examples, computing the task score includes computing a task familiarity score for one or more tasks completed by the user and computing a task difficulty score for the one or more tasks completed by the user. In some examples, the task familiarity score is based at least in part on a term frequency-inverse document frequency matrix representing words in a given task description. In some examples, the task difficulty score is based at least in part on a difference between an estimated effort to complete the one or more tasks completed by the user and an actual effort to complete the one or more tasks completed by the user. In some examples, determining the mood status includes receiving, from the task management service, a mood status manually selected by the user via a graphical user interface of the task management service. In some examples, determining the mood status includes receiving, from a smart device, health data representing a physiological indication of the user and/or a physical activity of the user. In some examples, the health data includes a sleep duration of the user.

Yet another example provides a system including a storage and at least one processor operatively coupled to the storage. The at least one processor is configured to execute instructions stored in the storage that when executed cause the at least one processor to carry out a process including receiving, from a task management service, one or more tasks to be performed by a user; computing a task score for each of the one or more tasks to be performed by the user; determining a mood status associated with the user; comparing the mood status to the task score for each of the one or more tasks to be performed by the user; determining, based on the comparison, a recommended task from among each of the one or more tasks to be performed by the user; and sending the recommended task to the task management service for display to the user. In some examples, computing the task score includes computing a task familiarity score for one or more tasks completed by the user and computing a task difficulty score for the one or more tasks completed by the user. In some examples, the task familiarity score is based at least in part on a term frequency-inverse document frequency matrix representing words in a given task description. In some examples, the task difficulty score is based at least in part on a difference between an estimated effort to complete the one or more tasks completed by the user and an actual effort to complete the one or more tasks completed by the user. In some examples, determining the mood status includes receiving, from the task management service, a mood status manually selected by the user via a graphical user interface of the task management service. In some examples, determining the mood status includes receiving, from a smart device, health data representing a physiological indication of the user and/or a physical activity of the user. In some examples, the health data includes a sleep duration of the user.

Other aspects, examples, and advantages of these aspects and examples, are discussed in detail below. It will be understood that the foregoing information and the following detailed description are merely illustrative examples of various aspects and features and are intended to provide an overview or framework for understanding the nature and character of the claimed aspects and examples. Any example or feature disclosed herein can be combined with any other example or feature. References to different examples are not necessarily mutually exclusive and are intended to indicate that a particular feature, structure, or characteristic described in connection with the example can be included in at least one example. Thus, terms like “other” and “another” when referring to the examples described herein are not intended to communicate any sort of exclusivity or grouping of features but rather are included to promote readability.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and are incorporated in and constitute a part of this specification but are not intended as a definition of the limits of any particular example. The drawings, together with the remainder of the specification, serve to explain principles and operations of the described and claimed aspects. In the figures, each identical or nearly identical component that is illustrated in various figures is represented by a like numeral. For purposes of clarity, not every component may be labeled in every figure.

FIG. 1 is a block diagram of a task management system, in accordance with an example of the present disclosure.

FIG. 2 is a block diagram of another task management system for recommending tasks to a user, in accordance with an example of the present disclosure.

FIGS. 3-7 show several example graphical user interfaces for use with the task management systems of FIGS. 1 and 2, in accordance with an example of the present disclosure.

FIG. 8 shows a task recommendation process, in accordance with an example of the present disclosure.

FIG. 9 is a flow diagram of a task management process, in accordance with an example of the present disclosure.

FIG. 10 is a block diagram of a computing device configured to implement various systems and processes in accordance with examples disclosed herein.

### DETAILED DESCRIPTION

Overview

As noted above, project management tools can be used for allocating resources, such as human labor, toward the completion of certain project goals. For example, a project team member may be assigned a series of tasks that are to be completed by a given deadline. However, it is often left up to the project team member to manage his or her own time on a day-to-day basis. In other words, each team member should decide for him or herself when to perform the assigned tasks in order to meet the deadlines set in the project management tools for those tasks. While existing project management tools are useful for identifying the tasks to be performed, they do not provide a sufficient level of detail to help project team members make decisions about which specific tasks to perform at a given moment.

For instance, the project team member may have many different tasks to perform, some of which are relatively easy and some of which are relatively difficult. Typically, these tasks are performed sequentially without much consideration of their difficulty levels. However, it has been found that work efficiency is a function of the worker's mental and biological states. For example, if a worker is not feeling well on a particular day, the worker will likely be less efficient at performing difficult tasks than easy tasks. Therefore, it may be better to delay the difficult tasks until the worker is feeling better.

To this end, a task management tool is disclosed. In some examples, the disclosed techniques evaluate several tasks to be performed by a user and suggest, to the user, which of the tasks should be performed presently (that is, during the current day) based on several factors, such as the level of difficulty for performing the task in view of the user's mood status. In some examples, the difficulty level of each task can be quantified in any number of ways, such as easy, normal, and difficult. In some examples, the user's mood status can be self-determined (that is, the user can select his or her mood from a list of moods presented via a graphical user interface) and/or based on biological data collected by a monitoring device, such as a smart watch or smartphone. The biological data can include, for example, information about the duration and quality of the user's sleep based on factors such as heartrate, breathing rate, blood pressure, and physical activity over a period of time. This approach provides an enhanced humanized working experience that more fully considers which of several tasks are more appropriate in view of the user's current mental and biological status.

In some examples, machine learning is utilized to implement algorithms for generating task suggestions based on the user's status. The disclosed techniques can be applied to any task management tool to provide humanized task suggestions.

Task Recommendation System

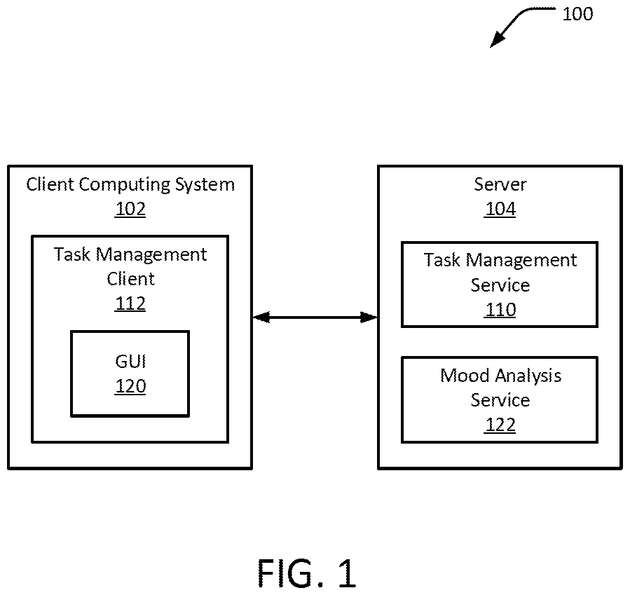

FIG. 1 is a block diagram of a task management system 100, in accordance with an example of the present disclosure. The task management system 100 includes a client computing system 102 and a server 104. The server 104 is configured to provide a task management service 110 and a mood analysis service 122 that support a task management client 112 executing on the client computing system 102. The task management client 112 includes a graphical user interface 120 that permits a user to interact with the task management client 112. The client computing system 102, the task management client 112, the server 104, and the task management service 110 can each be in communication with other components of the task management system 100 using, for example, a local area network, a wide area network, or any type of wired or wireless network.

In some examples, the server 104 can provide operational support for the task management service 110 and the mood analysis service 122, and communications support between the task management service 110, the mood analysis service 122, and the task management client 112. The task management service 110 can provide tools for individuals, teams, or organizations to complete projects by organizing, classifying, prioritizing, and recommending tasks to the end user(s). Additionally, the task management service 110 is configured to exchange data or otherwise interact with the mood analysis service 122. For example, as discussed in further detail below, the mood analysis service 122 can collect information about various tasks from one or more task management services, information about a user's mood status, and/or health data for the user and provide task recommendations to the user via the task management service 110 based on the task, mood, and/or health data.

Task Recommendation Process

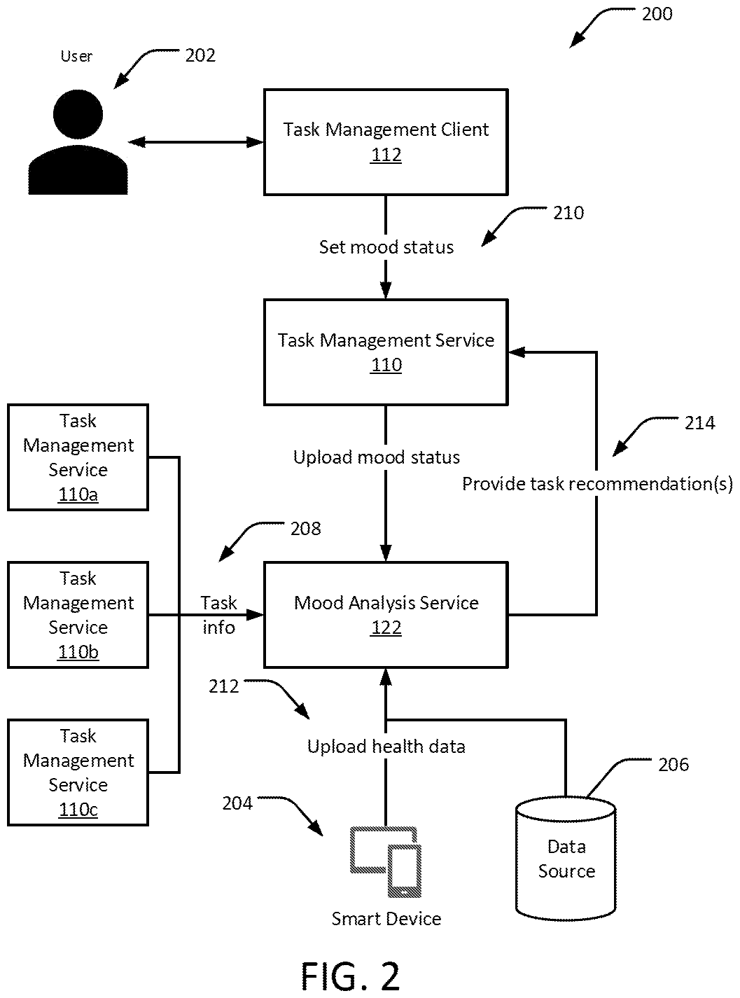

FIG. 2 is a block diagram of a system 200 for recommending tasks to a user 202, in accordance with an example of the present disclosure. The system includes at least portions of the task management system 100 of FIG. 1, including at least one task management client 112, at least one task management service 110, 110a-c, and at least one mood analysis service 122. For example, in one or more implementations of the system 200, the task management service 110a-c can include Wrike, Jira by Atlassian Corporation Plc of Sydney, Australia, or other project management software systems. In some examples, the system 200 further includes at least one smart device 204 and/or at least one data source 206 each configured to upload health data to the mood analysis service 122.

As noted above, the user 202 may have several different tasks 208, as specified by any of the task management services 110, 110a-c, that need to be completed. Often these tasks 208 are performed in tandem. Some of these tasks 208 may be more difficult to perform than others, and thus there is a natural inclination for the user to gravitate toward performing the easier tasks before the harder ones, particularly when the user is tired or not feeling well. However, the user 202 may not necessarily know the relative difficulty levels of the tasks 208 or how to sequence performance of the tasks 208 in the most efficient manner. For example, if the user 202 is feeling refreshed and well, it may be more efficient for the user 202 to perform the difficult tasks 208 before the easy tasks 208 to take advantage of his or her overall well being on a given day and reserve the easier tasks 208 for later in case the user is not feeling as well later on subsequent days. By completing the more difficult tasks 208 first, while the user 202 is feeling well, there is less risk that the user 202 becomes saddled with only difficult tasks 208 if he or she is not feeling as well on another day.

In some examples, the user 202 self-assesses his or her own mood and sets a corresponding mood status 210 in the task management service 110. For example, a positive mood status 210 indicates that the user 202 feels like he or she can do more work, or more difficult work, than usual, while a negative mood status 210 indicates that the user 202 is less inclined to perform difficult or stressful tasks. A neutral mood status 210 (or other suitable status indications) can be used to represent ranges of moods between positive and negative. The user's mood can be impacted by any number of factors, such as the user's emotional and/or physical health. For instance, the user 202 may have a positive mood when he or she is happy and energized, and a negative mood when he or she is frustrated or tired. The user 202 can set her or her mood status 210 at any time, typically once a day or several times a day if his or her mood changes during the course of the day.

In some other examples, the smart device 204 can collect health and physiological data 212 from the user 202 and use that data exclusively to automatically select the mood status 210. The smart device 204 can include any mobile computing device, such as a smartphone, a smart watch, a laptop computer, a tablet computer, or a thin client computer. Generally, the smart device 204 is battery powered for portability, but can nevertheless include devices that are not battery powered, such as small form factor desktop personal computers. The smart device 204 is configured to collect health and/or physiological data 212 from the user, either by monitoring biometric signals of the user 202 or by collecting data manually entered into the smart device 204 by the user, such as when the user 202 manually selects a mood status 210 or provides other information that can be used to evaluate the user's mood. In some examples, another data source 206, such as a database including data obtained from physiologic sensors, can provide the health and physiological data 212 to the mood analysis service 122.

The health and physiological data 212 can be used to interpret the user's mood automatically. For example, data representing sleep duration and quality can be collected from the smart device 204 to determine how refreshed or exhausted the user 202 may be on a given day. In another example, data representing physiological indications of the user 202 can be collected from the smart device 204 or from another data source 206 to determine the user's mood status. Such physiological indications can include, for example, blood pressure, oxygen saturation, heart rate, respiration, body temperature, and/or other measures of bodily functions and activity.

The mood analysis service 122 works in conjunction with the task management service 110 to identify pending tasks 208 that are most suitable for the user's current mood status 210. Based on the user's mood status 210, whether provided manually by the user 202 or determined automatically from health and physiological data 212, the mood analysis service 122 generates and provides task recommendations 214 to the user 202 via the task management service 110 to help improve the user's working experience while maintaining the highest possible efficiency or output.

Example Graphical User Interface

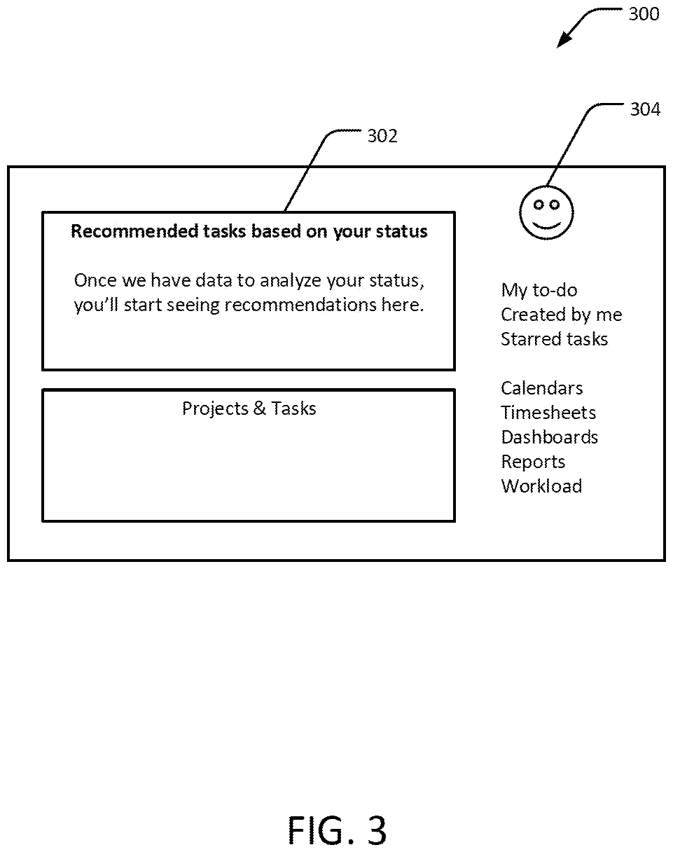

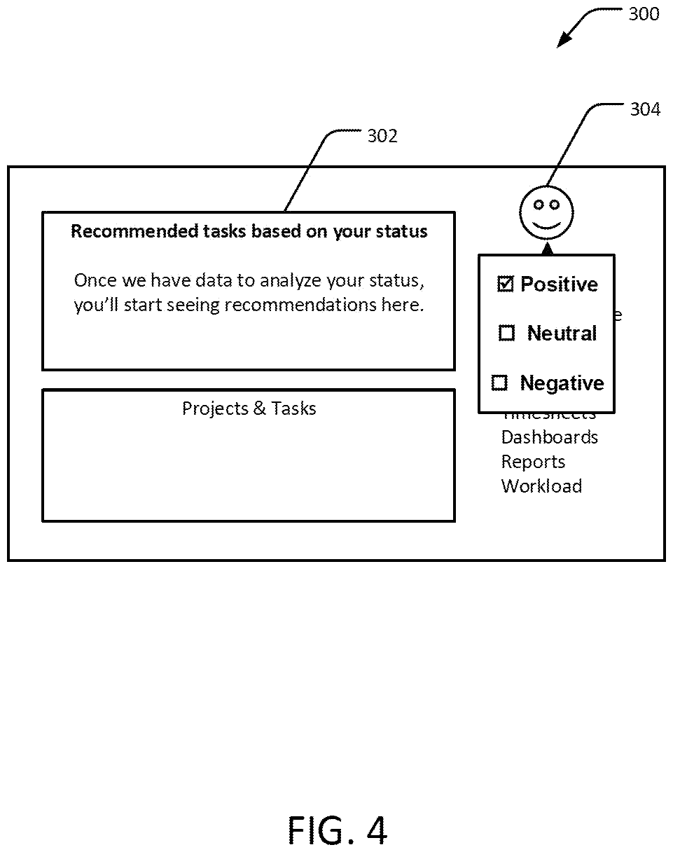

FIGS. 3-5 show an example graphical user interface (GUI) 300 for selecting a mood status via the task management service 110, in accordance with an example of the present disclosure. In some examples, the GUI 300 can be included within the task management service 110 and presented to the user 202 via the GUI 120 of the task management client 112. The GUI 300 includes a recommended task pane 302 and a mood status selection icon 304. The recommended task pane 302 is configured to display one or more tasks 208 that are recommended to the user 202 by the mood analysis service 122 based on the user's mood status 210. As noted above, in some examples the user 202 can manually select his or her mood status 210, such as positive mood, neutral mood, and negative mood, such as shown in FIG. 4, via a user input to the system (e.g., a mouse click or a keyboard entry).

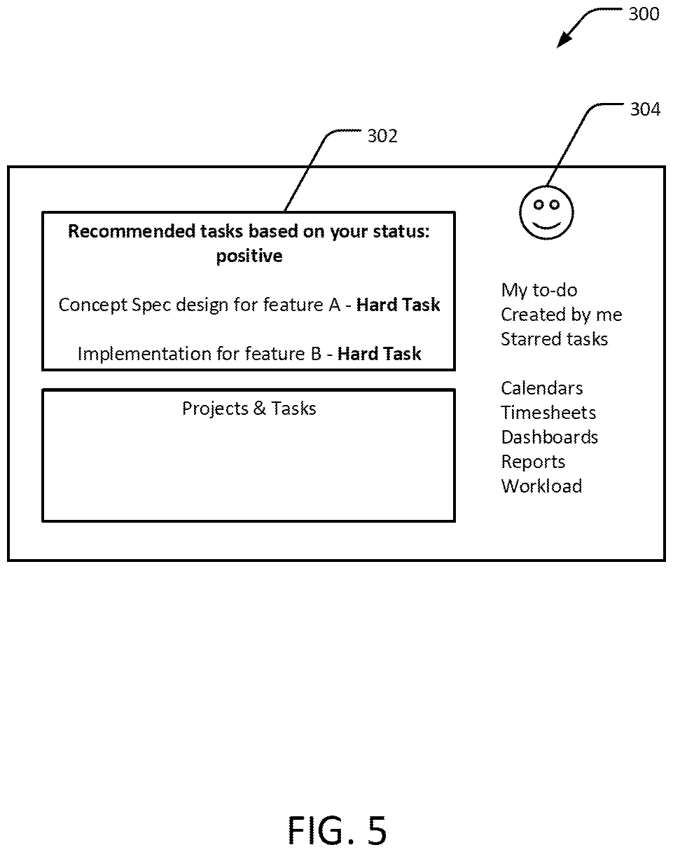

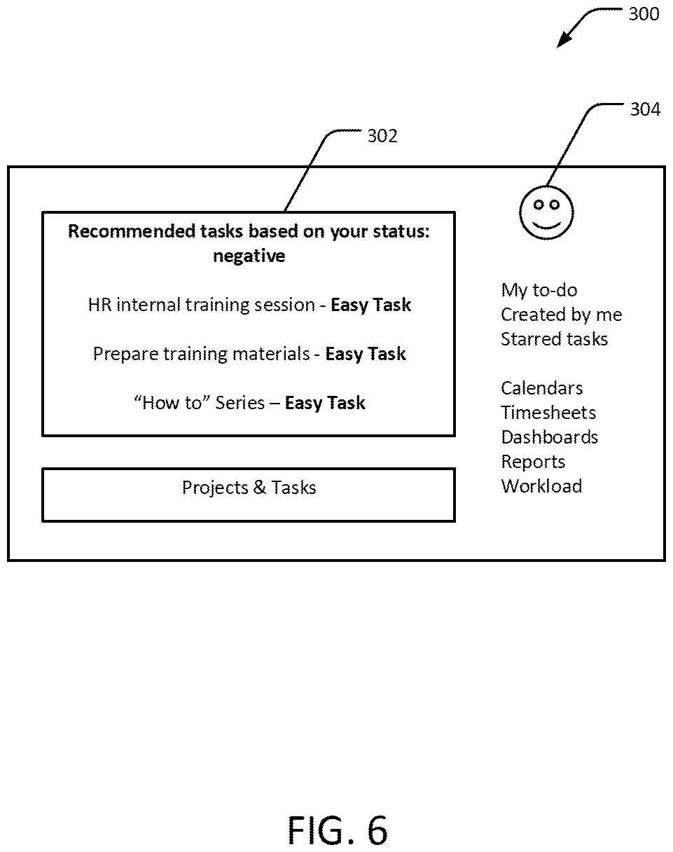

Once mood status 210 is set, the task management service 110 uploads the mood status 210 to the mood analysis service 122 for analysis. The result of the analysis influences which tasks 208 are to be suggested to the user 202. For example, if the user 202 manually selects a positive mood status 210, the mood analysis service 122 can provide recommendations of one or more difficult tasks 208 to the user in the recommended task pane 302, such as shown in FIG. 5. In another example, if the user 202 manually selects a negative mood status 210, the mood analysis service 122 can provide recommendations of one or more easy tasks 208 to the user 202 in the recommended task pane 302, such as shown in FIG. 6. In some examples, if there are multiple recommended tasks 208, the tasks 208 can be sorted based on the importance/urgency level such that the most important/urgent tasks 208 are listed first within the recommended task pane 302.

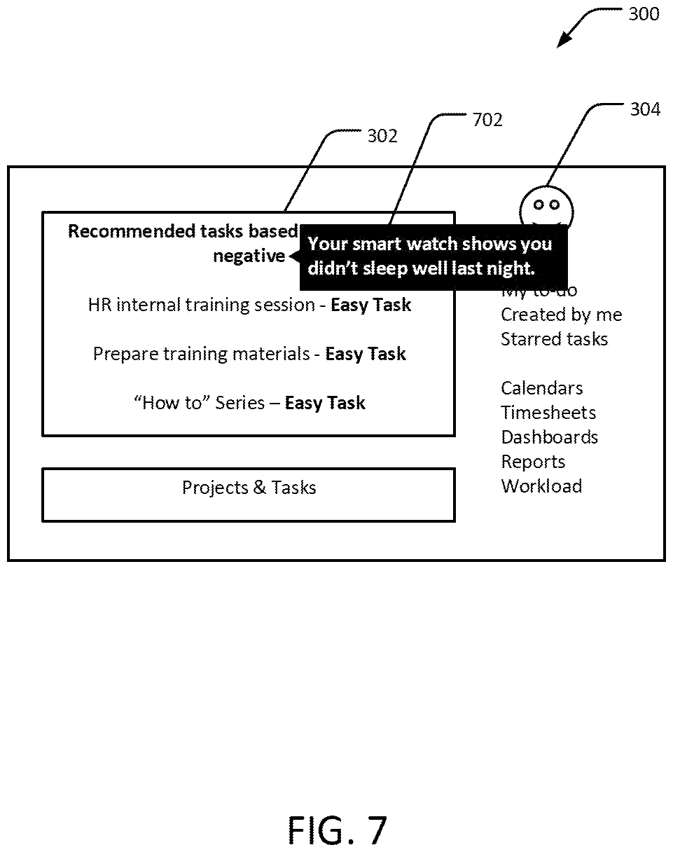

In some examples, if the user 202 does not manually select the mood status 210, the mood analysis service 122 can select the mood status 210 based on the health and physiological data 212, such as obtained from the smart device 204 or another data source 206. In such situations, the mood analysis service 122 can provide an explanation of the health and physiological data 212 via the GUI 300, such as shown in FIG. 7. For example, in FIG. 7, a pop-up dialog 702 is shown with an explanation that the user's smart watch detected that the user did not get enough sleep during the user's previous sleep cycle. Other such examples of explanations will be apparent in view of this disclosure. The explanation can help the user 202 understand how the mood analysis service 122 selected the mood status 210 using the health and physiological data 212, and additionally give the user 202 an opportunity to manually select the mood status 210 if the user 202 disagrees with or otherwise wishes to change the mood status 210 automatically selected by the mood analysis service 122.

Mood Analysis Service

The mood analysis service 122 is configured to receive the health and physiological data 212 from the smart device 204 or another data source 206 and to select the mood status 210. The mood analysis service 212 also collects task information 208 from one or more task management service 110, 110a-c, including a name or other description of each task and a status of each task, such as task complete, task in progress, and task not yet started. The mood analysis service 122 recommends one or more incomplete tasks 214 based on the mood status 210.

As noted above, the mood status 210 can be selected manually by the user (e.g., via the GUI 300 of FIG. 4) or automatically (e.g., using the health and physiological data 212 from the smart device 204 or other data source 206). For example, the user 202 can select the mood status 210 manually via a GUI 300 of the task management service 110, such as shown in FIG. 4. In another example, the mood analysis service 122 can collect available health data from the smart device 204, including sleep data, blood oxygen content data, workout/activity data, etc., and select the mood status 210 automatically using machine learning models and/or multi-classification algorithms such as a Naïve Bayes classifier for training the machine learning model to classify the health data 212 into one of the mood status categories (e.g., positive, neutral, and negative), according to the following equation:

$$P ( H \vert E ) = \frac{P ( E \vert H ) * P ( H )}{P ( E )}$$

where H represents a set of classes or categories (e.g., positive mood, neutral mood, and negative mood), where E represents a vector of features (evidence represented by independent variables in the health and/or physiological data), and where P represents the conditional probability that the features belong to a given class within the set of classes H. Since P(E) does not depend on H (the set of possible classes), it is effectively constant, and the classifier thus produces a joint probability (expectation) that the health and/or physiological data E falls into one of the possible classes H (e.g., positive mood, neutral mood, or negative mood).

In some examples, the user 202 can provide feedback for retraining the machine learning model. For example, if the mood analysis service 122 selects a mood status 210 of positive when the user 202 is feeling a negative mood, the user 202 can manually change the mood status 210 from positive to negative via the GUI 300. The mood analysis service 122 then uses the manual change of mood status 210 to retrain the machine learning model and/or provide the task recommendation(s) 214.

In another example, the mood analysis service 122 can collect available health data 212 from the smart device 204, including sleep data, blood oxygen content data, workout data, etc., and select the mood status 210 automatically using other algorithms to classify the health data 212 into one of the mood status categories (e.g., positive, neutral, and negative). For example, if the health data 212 includes the number of hours of sleep of the user, and 7-9 hours is a recommended sleep time duration for an average adult, the mood analysis service 122 can set the mood status 210 based on how many hours of sleep the user 202 got the previous night. In this example, if the sleep time duration is less than 7 hours, the mood status 210 is set to negative; if the sleep time duration is between 7 and 9 hours, the mood status 210 is set to neutral; and if the sleep time duration is greater than 9 hours, the mood status 210 is set to positive. The user 202 may manually change the mood status 210 after the mood analysis service 122 automatically selects the mood status 210.

If multiple techniques are used to automatically select the sleep status (e.g., machine learning and another algorithm, such as the sleep time algorithm described above) and each technique arrives at a different mood status, the lowest mood status is selected. For example, if machine learning determines that the mood status 210 is neutral or positive and another algorithm determines that the mood status 210 is negative, the mood status 210 will be set to negative.

Task Classification

In some examples, the mood analysis service 122 classifies each task 208 into one of several difficulty levels, such as difficult, normal, and easy. These classifications are used by the mood analysis service 122 to recommend tasks 214 based on the user's mood status 210. For example, the mood analysis service 122 can recommend easy tasks 214 when the user's mood status 210 is negative, normal tasks 214 when the user's mood status 210 is neutral, and difficult tasks 214 when the user's mood status 210 is positive.

Task Familiarity Score

In some examples, the difficulty level of a given task 208 can be determined based on the user's activity or familiarity with the task 208, including a difference between the user's estimation of how long it will take to complete the task 208 and the actual amount of time needed to complete a similar task 208.

Machine learning can be used to calculate a familiarity score for each task 208 the user 202 is about to work on (e.g., to produce a familiarity score between 0 and 1). The mood analysis service 122 maintains a list of all tasks 208 competed by the user 202 along with a task summary. For each completed task 208, the task summary is generated as follows. First, the mood analysis service 122 tokenizes task descriptions (e.g., sentences describing the task or words in a document associated with the task). Next, the mood analysis service 122 creates a term frequency matrix TF(t) of the words in each task description (e.g., values representing the number of times each word appears in the task description) as follows:

$$TF ( t ) = \frac{Number of times term t appears in a document}{Number of terms in the document}$$

Next, the mood analysis service 122 creates a table of documents ordered by word by calculating an inverse document frequency as follows:

$$IDF ( t ) = log ( \frac{Total number of documents}{Number of documents with term t} )$$

The mood analysis service 122 generates a term frequency-inverse document frequency matrix of words in the task descriptions as follows:

TF−IDF(t)=TF(t)*IDF(*)

Next, the mood analysis service 122 scores each sentence and calculates a threshold value based on the average sentence score. The mood analysis service 122 then selects sentences having a score greater than the average score and merges the selected sentences together to form the task summary.

The mood analysis service 122 calculates a familiarity score, referred to herein as task_{familiarity}, between a new task and one or more existing tasks in the list using a text similarity algorithm (e.g., Cosine similarity algorithm as shown below), where A and B represent the new task and the one or more existing tasks, respectively:

$$cos ( θ ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$

Based on the result, only those tasks 208 with low similarity score will be added into the list.

For in-progress tasks 208, the mood analysis service 122 calculates familiarity scores as described above and uses the task 208 with the highest familiarity score for the recommended task 214.

Task Difficulty Score

When there is a difference between the user's estimation of task difficulty and the actual effort spent on similar tasks, the mood analysis service 122 can use the familiarity score for similar tasks completed by all users to classify the difference as failed to meet estimated effort, met estimated effort, and ahead of estimated effort, and then calculate a percentage for each class. If most people (the largest percentage among all classes) failed to meet the estimated effort and spent more time to finish the similar task, the mood analysis service 122 classifies the task 208 as difficult. If most people (the largest percentage among all classes) met the estimated effort for the similar task, the mood analysis service 122 classifies the task 208 as normal. If most people (the largest percentage among all classes) completed the similar task ahead of the estimated effort (e.g., required less time to complete the task than estimated), the mood analysis service 122 classifies the task 208 as easy.

The mood analysis service 122 classifies the difficulty of the task 208 as follows:

task_{difficulty}=max(P_{fail},Pₘₑₜ,P_{ahead})

The mood analysis service 122 can tune or weight coefficients (e.g., using machine learning) for the task familiarity value and the task difficulty value then provide recommended task(s) 214 to the user 202 based on a task score as follows:

task_{score}=α*task_{familiarity}+β*task_{difficulty}

In an example, for the final task score 0.7 can be chosen as a threshold value to determine the difficulty level. For example, the task 208 is difficult if the task score<0.7, the task 208 is normal if the task score=0.7, and the task 208 is easy if the task score>0.7. The user 202 can provide his/her feedback to the system to modify the coefficients in the task score equation above.

In another example, the user 202 can manually select the task difficulty level.

In some examples, the mood analysis service 122 matches the mood status 210 with the task difficulty level as follows:

| Status | Difficulty level for tasks |
|---|---|
| Positive | Difficult |
| Neutral | Normal |
| Negative | Easy |

If the user's status is positive, the mood analysis service 122 recommends a difficult task 214; if the user's status is neutral, the mood analysis service 122 recommends a normal task 214; and if the user's status is negative, the mood analysis service 122 recommends an easy task 214. If there are multiple recommended tasks 214 (e.g., there are two or more difficult tasks recommended to the user), the mood analysis service 122 can further sort the tasks 214 based on the respective task scores and/or according to the corresponding importance/urgency level of each task 214, such that the most important or urgent tasks 214 are listed first.

Task Recommendation

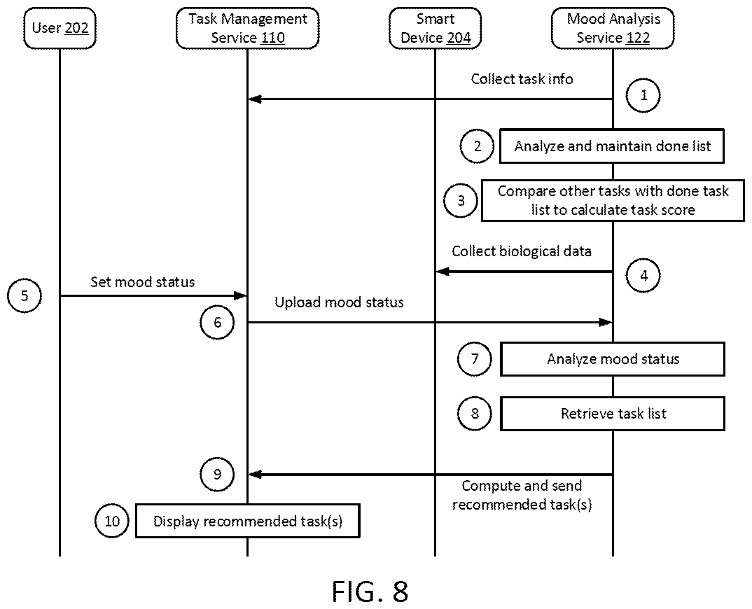

FIG. 8 shows a task recommendation process, in accordance with an example of the present disclosure. The mood analysis service 122 can be provided on the server 104, such as a cloud server, and be configured to collect task information 208 from the task management service 110, 110a-c, as indicated at step 1. The task information 208 can be collected by the mood analysis service 122 periodically, for example, daily, or at other intervals. Next, the mood analysis service 122 analyzes and maintains the completed task list, as indicated at step 2, such as described above to classify the difficulty of incomplete tasks. The mood analysis service 122 calculates the task score and stores it along with the task description, as indicated at step 3, by comparing incomplete tasks with completed tasks.

The mood analysis service 122 collects health data 212 from the smart device 204 or other data source 206, as indicated at step 4. The health data 212 can be collected at periodic intervals or on demand. For example, the user 202 can select the mood status 210 manually within the task management service 110, as indicated at step 5 and such as described with respect to FIG. 4, and the task management service 110 uploads the mood status 210 to the mood analysis service 122, as indicated at step 6.

If the mood status 210 exists from either the task management service 110 or the smart device 204, the mood analysis service 122 analyzes the mood status 210, as indicated at step 7, and then retrieves the task information 208 collected at step 1 for the user, as indicated at step 8. The mood analysis service 122 then calculates the recommended task or tasks 214, such as described above, and sends the recommended task(s) 214 to the task management service 110, as indicated at step 9. The task management service 110 then displays the recommended tasks to the user, as indicated at step 10, via the GUI 120 of the task management client 112.

Example Task Management Process

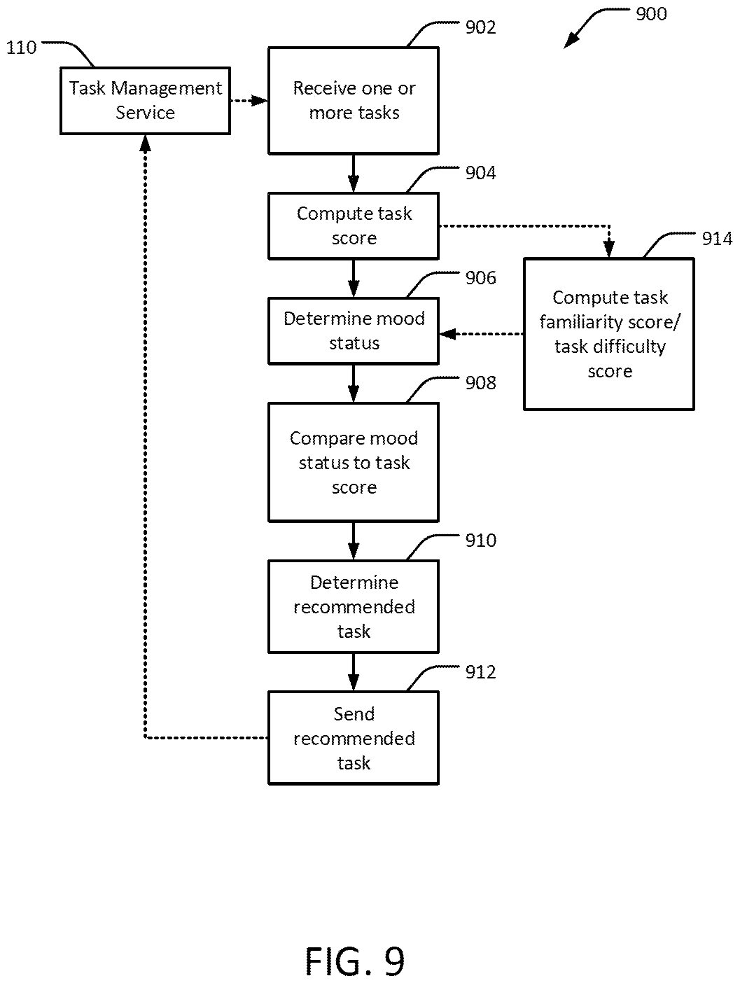

FIG. 9 is a flow diagram of a task management process 900, in accordance with an example of the present disclosure. The process 900 can be implemented, for example, by the task management system 100 of FIG. 1 and at least partially within the mood analysis service 122. The process 900 includes receiving 902, from a task management service, one or more tasks to be performed by a user. The tasks can be obtained from one or multiple task management services and can include any tasks assigned to the user that have not been completed. The process 900 further includes computing 904 a task score for each of the one or more tasks to be performed by the user. For example, a task score can be computed 914 for each task based on a task familiarity score and a task difficulty score, such as described above. In some examples, the task familiarity score is based at least in part on a term frequency-inverse document frequency matrix representing words in a given task description (e.g., words associated with the task or words in documents related to the task). In some examples, the task difficulty score is based at least in part on a difference between an estimated effort to complete the one or more tasks completed by the user and an actual effort to complete the one or more tasks completed by the user.

The process 900 further includes determining 906 a mood status associated with the user. The mood status is a value that represents the mood of the user at a given time, such as a positive mood, a neutral mood, or a negative mood. In some examples, determining the mood status includes receiving, from the task management service, a mood status manually selected by the user via a graphical user interface of the task management service, such as described with respect to FIG. 4. In some examples, determining the mood status includes receiving, from a smart device, health data representing a physiological indication of the user and/or a physical activity of the user, such as described with respect to FIG. 8. For example, the health data provided by the smart device includes a sleep duration of the user, such as described above. The process 900 further includes comparing 908 the mood status to the task score for each of the one or more tasks to be performed by the user and determining 910, based on the comparison, a recommended task from among each of the one or more tasks to be performed by the user. For example, for the task score 0.7 can be chosen as a threshold value to determine the difficulty level. The task is difficult if the task score<0.7, the task is normal if the task score=0.7, and the task is easy if the task score>0.7. The recommended task is one or more of the tasks to be performed by the user having a difficulty level corresponding to the mood status, such as easy tasks for a negative mood status, normal tasks for a neutral mood status, and difficult tasks for a positive mood status. The process 900 further includes sending 912 the recommended task to the task management service 110 for display to the user, such as shown in FIGS. 5 and 6.

Example Computing Device

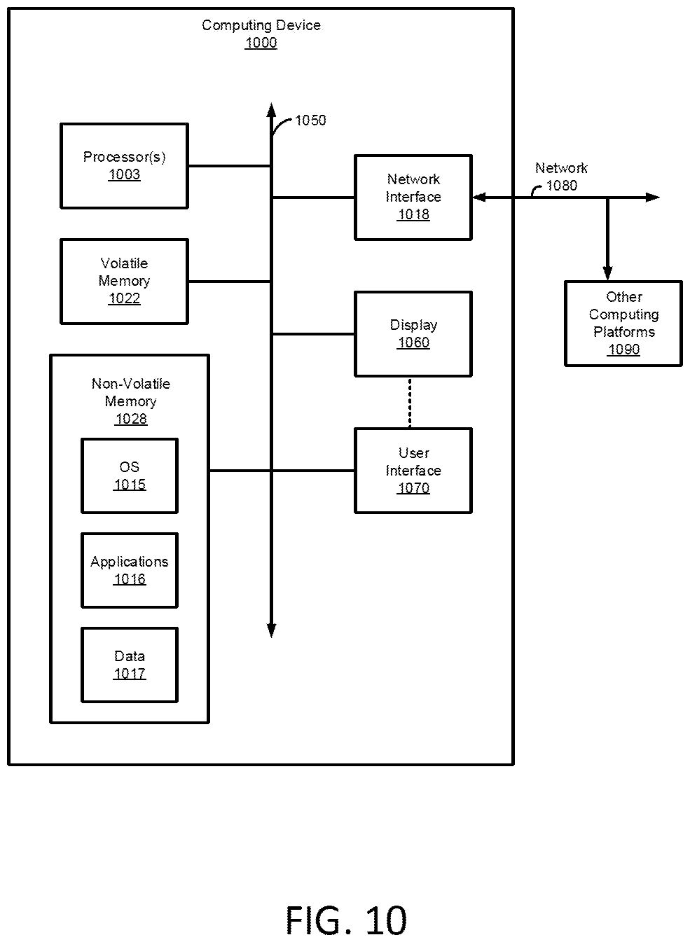

FIG. 10 is a block diagram of a computing device 1000 configured to implement various systems and processes in accordance with examples disclosed herein. It will be understood that multiple computing devices 1000 can be implemented according to the examples provided herein, where each of the computing devices 1000 is configured to perform certain functions in conjunction with other computing devices 1000. In some examples, the computing device 1000 can include a workstation, a laptop computer, a tablet, a mobile device, or any suitable computing or communication device. One or more components of the computing device 1000, including the client computing system 102, the server 104, and/or the smart device 204, can include or otherwise be executed using one or more processors 1003, volatile memory (e.g., random access memory (RAM)) 1022, non-volatile machine-readable mediums (e.g., a non-volatile memory 1028), one or more network or communication interfaces 1018, a display 1060, a user interface (UI) 1070, and a communications bus 1050. The non-volatile (non-transitory) machine-readable mediums 1028 can include: one or more hard disk drives (HDDs) or other magnetic or optical machine-readable storage media; one or more machine-readable solid state drives (SSDs), such as a flash drive or other solid-state storage media; one or more hybrid machine-readable magnetic and solid-state drives; and/or one or more virtual machine-readable storage volumes, such as a cloud storage, or a combination of such physical storage volumes and virtual storage volumes or arrays thereof. The user interface 1070 can include one or more input/output (I/O) devices (e.g., a mouse, a keyboard, a touch screen/monitor/panel, a microphone, one or more speakers, etc.). The display 1060 can provide a graphical user interface (GUI). The non-volatile memory 1028 stores an operating system (OS), one or more applications, and data such that, for example, computer instructions of the operating system and the applications, are executed by processor(s) out of the volatile memory. In some examples, the volatile memory 1022 can include one or more types of RAM and/or a cache memory that can offer a faster response time than a main memory. Data can be entered through the user interface 1070. Various elements of the computing device 1000 can communicate via the communications bus 1050 or the network interface 1018.

The computing device 1000 can also be referred to as a client device, a computing device, an endpoint device, a computer, a computer system, or a server. The computing device 1000 is shown as an example client computing system 102, server 104, and/or smart device 204, and can be implemented within any computing or processing environment with any type of physical or virtual machine or set of physical and virtual machines that can have suitable hardware and/or software capable of operating as described herein. In some examples, some components of the computing device can be implemented virtually (e.g., using a combination of hardware and software), such as to provide GUI 120 to the task management client 112 of the client computing system 102, where the task management service 110 emulates certain processing functions of the client computing system 102 (e.g., including at least portions of the method 900 of FIG. 9) using hardware components of the client computing system 102 (e.g., processors, network communications hardware, I/O devices, etc.).

The non-volatile memory 1028 stores an operating system (OS) 1015, one or more applications or programs 1016, and data 1017. The OS 1015 and the applications 1016 include sequences of instructions that are encoded for execution by processor(s) 1003. Execution of these instructions results in manipulated data. Prior to their execution, the instructions can be copied to the volatile memory 1022. In some examples, the volatile memory 1022 can include one or more types of RAM and/or a cache memory that can offer a faster response time than a main memory. Data can be entered through the user interface 1070 or received from the other I/O device(s), such as the network interface 1018. The various elements of the computing device 1000 described above can communicate with one another via the communications bus 1050 and/or via the network interface 1018 to other computing platforms 1090.

The processor(s) 1003 can be implemented by one or more programmable processors to execute one or more executable instructions, such as a computer program, to perform the functions of the system. As used herein, the term “processor” describes circuitry or hardware that performs a function, an operation, or a sequence of operations. The function, operation, or sequence of operations can be hard coded into the circuitry or a data storage device, or soft coded by way of instructions held in the storage device and executed by the circuitry. A processor can perform the function, operation, or sequence of operations using digital values and/or using analog signals. In some examples, the processor can include one or more application specific integrated circuits (ASICs), microprocessors, digital signal processors (DSPs), graphics processing units (GPUs), microcontrollers, field programmable gate arrays (FPGAs), programmable logic arrays (PLAs), multicore processors, or general-purpose computers with associated memory. The processor(s) 1003 can be analog, digital, or a combination of these. In some examples, the processor(s) 1003 can be one or more local physical processors or one or more remotely located physical processors. A processor including multiple processor cores and/or multiple processors can provide functionality for parallel, simultaneous execution of instructions or for parallel, simultaneous execution of one instruction on more than one piece of data.

The network interfaces 1018 can include one or more interfaces to enable the computing device 1000 to access a computer network 1080 such as a Local Area Network (LAN), a Wide Area Network (WAN), a Personal Area Network (PAN), or the Internet through a variety of wired and/or wireless connections, including cellular connections and Bluetooth connections. In some examples, the network 1080 may allow for communication with other computing devices 1090, such as the client computing device 102, the server 104, and/or the smart device 204, to enable distributed, shared, or cooperative computing (e.g., such as cooperatively functioning to enable and provide enable MFA to one or more of the applications 1016). The network 1080 can include, for example, one or more private and/or public networks over which computing devices can exchange data.

In described examples, the computing device 1000 can execute an application on behalf of a user of the client computing system 102. For example, the computing device 1000 can execute one or more virtual machines managed by a hypervisor. Each virtual machine can provide an execution session within which applications execute on behalf of a user or a client device, such as a hosted desktop session. The computing device 1000 can also execute a terminal services session to provide a hosted desktop environment. The computing device 1000 can provide access to a remote computing environment including one or more applications, one or more desktop applications, and one or more desktop sessions in which one or more applications can execute.

The foregoing description and drawings of various embodiments are presented by way of example only. These examples are not intended to be exhaustive or to limit the present disclosure to the precise forms disclosed. Alterations, modifications, and variations will be apparent in light of this disclosure and are intended to be within the scope of the present disclosure as set forth in the claims. For example, the processes disclosed herein each represent a sequence of acts in a particular example. Some acts are optional and, as such, can be omitted in accord with one or more examples. Additionally, the order of acts can be altered, or other acts can be added, without departing from the scope of the apparatus and methods discussed herein.

Also, the phraseology and terminology used herein is for the purpose of description and should not be regarded as limiting. Any references to examples, components, elements or acts of the systems and methods herein referred to in the singular can also embrace examples including a plurality, and any references in plural to any example, component, element or act herein can also embrace examples including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” can be construed as inclusive so that any terms described using “or” can indicate any of a single, more than one, and all of the described terms. In addition, in the event of inconsistent usages of terms between this document and documents incorporated herein by reference, the term usage in the incorporated references is supplementary to that of this document; for irreconcilable inconsistencies, the term usage in this document controls.

What is claimed is:

1. A task management method comprising:
receiving, by a processor and from a task management service, one or more tasks to be performed by a user;
computing, by the processor, a task score for each of the one or more tasks to be performed by the user;
determining, by the processor, a mood status associated with the user;
comparing, by the processor, the mood status to the task score for each of the one or more tasks to be performed by the user;
determining, by the processor and based on the comparison, a recommended task from among each of the one or more tasks to be performed by the user; and
sending, by the processor, the recommended task to the task management service for display to the user.

2. The method of claim 1, wherein computing the task score includes computing a task familiarity score for one or more tasks completed by the user and computing a task difficulty score for the one or more tasks completed by the user.

3. The method of claim 2, wherein the task familiarity score is based at least in part on a term frequency-inverse document frequency matrix representing words in a given task description.

4. The method of claim 2, wherein the task difficulty score is based at least in part on a difference between an estimated effort to complete the one or more tasks completed by the user and an actual effort to complete the one or more tasks completed by the user.

5. The method of claim 1, wherein determining the mood status includes receiving, from the task management service, a mood status manually selected by the user via a graphical user interface of the task management service.

6. The method of claim 1, wherein determining the mood status includes receiving, from a smart device, health data representing a physiological indication of the user and/or a physical activity of the user.

7. The method of claim 6, wherein the health data includes a sleep duration of the user.

8. A computer program product including one or more non-transitory machine-readable mediums having instructions encoded thereon that when executed by at least one processor cause a process to be carried out, the process comprising:
receiving, from a task management service, one or more tasks to be performed by a user;
computing a task score for each of the one or more tasks to be performed by the user;
determining a mood status associated with the user;
comparing the mood status to the task score for each of the one or more tasks to be performed by the user;
determining, based on the comparison, a recommended task from among each of the one or more tasks to be performed by the user; and
sending the recommended task to the task management service for display to the user.

9. The computer program product of claim 8, wherein computing the task score includes computing a task familiarity score for one or more tasks completed by the user and computing a task difficulty score for the one or more tasks completed by the user.

10. The computer program product of claim 9, wherein the task familiarity score is based at least in part on a term frequency-inverse document frequency matrix representing words in a given task description.

11. The computer program product of claim 9, wherein the task difficulty score is based at least in part on a difference between an estimated effort to complete the one or more tasks completed by the user and an actual effort to complete the one or more tasks completed by the user.

12. The computer program product of claim 8, wherein determining the mood status includes receiving, from the task management service, a mood status manually selected by the user via a graphical user interface of the task management service.

13. The computer program product of claim 8, wherein determining the mood status includes receiving, from a smart device, health data representing a physiological indication of the user and/or a physical activity of the user.

14. The computer program product of claim 13, wherein the health data includes a sleep duration of the user.

15. A system comprising:
a storage; and
at least one processor operatively coupled to the storage, the at least one processor configured to execute instructions stored in the storage that when executed cause the at least one processor to carry out a process comprising
receiving, from a task management service, one or more tasks to be performed by a user;
computing a task score for each of the one or more tasks to be performed by the user;
determining a mood status associated with the user;
comparing the mood status to the task score for each of the one or more tasks to be performed by the user;
determining, based on the comparison, a recommended task from among each of the one or more tasks to be performed by the user; and
sending the recommended task to the task management service for display to the user.

16. The system of claim 15, wherein computing the task score includes computing a task familiarity score for one or more tasks completed by the user and computing a task difficulty score for the one or more tasks completed by the user.

17. The system of claim 16, wherein the task familiarity score is based at least in part on a term frequency-inverse document frequency matrix representing words in a given task description.

18. The system of claim 16, wherein the task difficulty score is based at least in part on a difference between an estimated effort to complete the one or more tasks completed by the user and an actual effort to complete the one or more tasks completed by the user.

19. The system of claim 15, wherein determining the mood status includes receiving, from the task management service, a mood status manually selected by the user via a graphical user interface of the task management service.

20. The system of claim 15, wherein determining the mood status includes receiving, from a smart device, health data representing a physiological indication of the user and/or a physical activity of the user.
